(19) United States
(12) Patent Application Publication
(10) Pub. No.: US 2021/0299464 A1
(43) Pub. Date: Sep. 30, 2021

(54) PHOTOTHERAPEUTIC DEVICE FOR A PENIS AND A USING METHOD THEREOF

(71) Applicant: Vivienne Reign, Irvine, CA (US)

(72) Inventor: Vivienne Reign, Irvine, CA (US)

(21) Appl. No.: 16/834,770

(22) Filed: Mar. 30, 2020

(51) Int. Cl. A61N 5/06

(52) U.S. Cl. CPC A61N 5/0613; A61N 2005/0652; A61N 2005/0659; A61N 2005/0626; A61N 2005/0663; A61N 2005/0645

(57) ABSTRACT

A phototherapeutic device for a penis and a using method thereof can include a base material, a light emitting element and means for fixing or attaching the base material to substantially conform to the shape of a body part. The light emitting element can include a plurality of LED lights that can generate light at various wavelengths. Some embodiments can also include a controller to control the light emitting element. The invention also includes methods of using a phototherapeutic device to deliver wavelengths of light to at least a portion of a body part.

### BACKGROUND OF THE INVENTION

### a) Field of the Invention

The present invention relates to a phototherapeutic device for a penis and a using method thereof.

### b) Description of the Prior Art

The use of light waves to treat multiple disorders within Humans is well known and medically documented through multiple clinical studiers. Light waves can provide cosmetic and therapeutic benefits to Human.

### SUMMARY OF THE INVENTION

Embodiments of the present invention are directed to devices that can deliver light waves or other electromagnetic radiation waves to as a human, or parts of an Human. Embodiments also include methods of assembling and using such devices. In some embodiments, the device includes a flexible base material and a light emitting element that can include an array of light-emitting diodes (LED). Embodiments can also include an electronic controller for controlling the light emitting element and means to attach or fix the base material to substantially conform to the shape of a body part. In some embodiments, the device can be applied or used for a penis. Embodiments can be used to generate light in various wavelengths to treat or improve blood circulation, which can improve a man's sexual ability and health, as well as to treat symptoms of erectile dysfunction.

In some embodiments of the present invention, the means to attach or fix the flexible material includes the combination of magnets and metallic pieces.

Some embodiments include a belt for connecting or attaching the device to the treatment area.

Some embodiments include a translucent element partially or completely disposed on or over the light emitting element.

In some embodiments, the wavelengths of light delivered by the device range from about 600 nm to about 900 nm, RED LED (wavelength 625 nm-680 nm) and INFRA RED (wavelength 810 nm-910 nm).

In some embodiments, the base material is silica gel.

Other embodiments of the present invention relate to methods of using a phototherapeutic device. These exemplary methods can include providing a device that includes a flexible base material, a light emitting element, an electronic controller for controlling the element, and means for attaching or fixing the base material to substantially conform to the shape of a body part.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a three-dimensional schematic view of a preferred embodiment of the present invention.

FIG. 2 shows a three-dimensional exploded view of the preferred embodiment of FIG. 1.

FIG. 3 shows a schematic view of a preferred embodiment of the present invention that is connected or attached to a human.

FIG. 4 shows a schematic view of a preferred embodiment of the present invention that is attached or fixed to substantially conform to the shape of a body part.

### DETAILED DESCRIPTION OF THE PREFERRED EMBODIMENT

The following examples and descriptions illustrate embodiments of the present invention. One who is skilled in the art disclosed in the present specification may easily understand other advantages and effects of the present invention.

In the accompanying drawings, the structure illustrated, the ratio, and the size, etc., are used only with the contents disclosed in the specification for reading and understanding by those who are skilled in the art, and are not to be intended to limit the conditions of implementation. The words cited in this specification as “one,” “two,” and “upper,” etc., are merely for illustrative purpose, and are not to be intended to limit the range of implementation of the present invention; the change or adjustment in their relative relationships is also considered as the scope of the implementation of the present invention, if there is no substantial change in the technology contents.

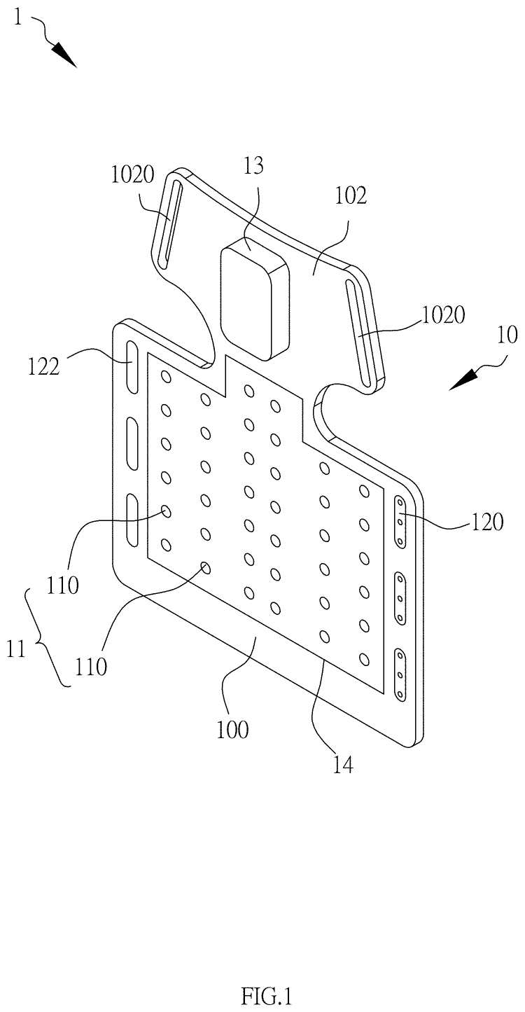

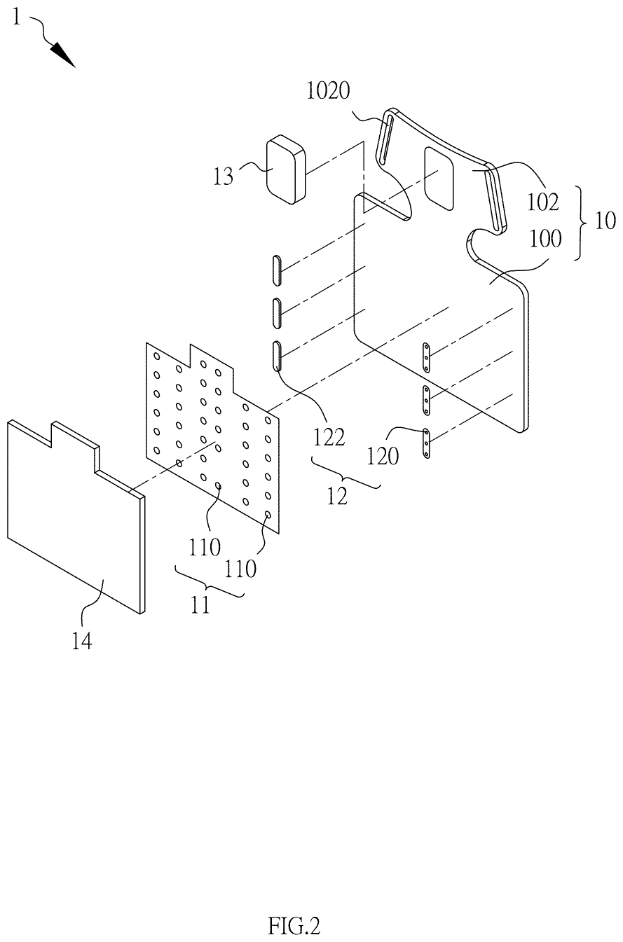

FIG. 1 and FIG. 2 show preferred embodiments of the present invention. Embodiments of the present invention include a device for treating an animal, such as a human, or human body parts. The body part is not limited and includes, an arm or parts of an arm, a leg or parts of a leg, an ankle, a neck, and a penis. In particularly preferred embodiments, the body part is a penis.

A preferred embodiment device 1 comprises a flexible base material 10, a light emitting element 11, means for attaching or fixing the device 12, and a controller 13, wherein the base material 10 is preferably silica gel and is configured in a shape that enables it to substantially conform to the shape of a body part. The shape of the base material is not limited and can include any shape that can be fixed or attached to conform to the shape of a body part, such as a leg, an ankle, an arm, a neck, and a penis. In some embodiments, the shape is square, in other embodiments the shape is rectangular or circular. The base material need not be flexible and can be an inflexible or semiflexible material. In some embodiments, the base material is plastic. In some embodiments, the base material is a molded plastic material. In some embodiments, the base material is molded to conform to the shape of a body part, such as an arm, a leg, an ankle, a neck, or a penis.

Embodiments can include an enclosing portion 100, which can be attached or fixed to enclose a body part, and a fixing portion 102, which can include slots or holes or other means to attach a belt or attachment mechanism to connect or attach the device to a human. The enclosing portion 100 can include light emitting element 11. Light emitting element 11 can be a flexible material and can include multiple LED lights 110 that generate light in various wavelengths, including between about 600 nm and 900 nm, RED LED (wavelength 625 nm-680 nm) and INFRA RED (wavelength 810 nm-910 nm). Moreover, the wavelengths of light can be applied in various sequences and patterns, as well as for pre-defined lengths of time. The light emitting element 11 can be a flexible material in a rectangular or any other shape with several rows of LED lights.

The number of LED lights is not limited and can be any number between about 4 and about 100 lights. The arrangement of LED lights is also not limited and can include rows of lights, as well as other patterns, such as circular patterns, rectangular patterns, and sunburst patterns. The number and arrangement of lights can differ depending on the body part for treatment.

In some embodiments, the LED lights 110 are connected to and controlled by the controller 13, and the controller 13 can be partially or completely disposed on the fixing portion 102.

In some embodiments, means for attaching or fixing the device 12 includes magnets 120 and metallic pieces 122. The attaching or fixing means are not limited to magnets and metallic pieces, and can include other things such as hooks, buttons, Velcro, loops, and adhesives. The means for attaching or fixing the device 12 can be structured to allow a user to adjust the device. In some embodiments, the magnets 120 can be disposed on one side of the enclosing portion 100, and the metallic pieces 122 can be disposed on the other side of the enclosing portion 100.

In some embodiments, the device can also include slots or holes 1020 that can be configured to accept a belt. The belt can be used to connect or attach the device to an animal. Some embodiments can also include a translucent element 14 the can be partially or completely disposed on or over the enclosing portion 100 and cover part or all of the light emitting element 11. The translucent material can be TPE (Thermoplastic Elastomer), TPU (Thermoplastic Polyurethane) or other similar material.

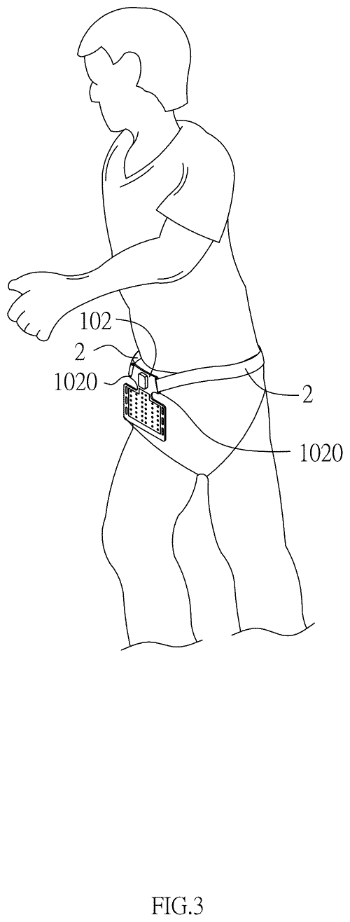

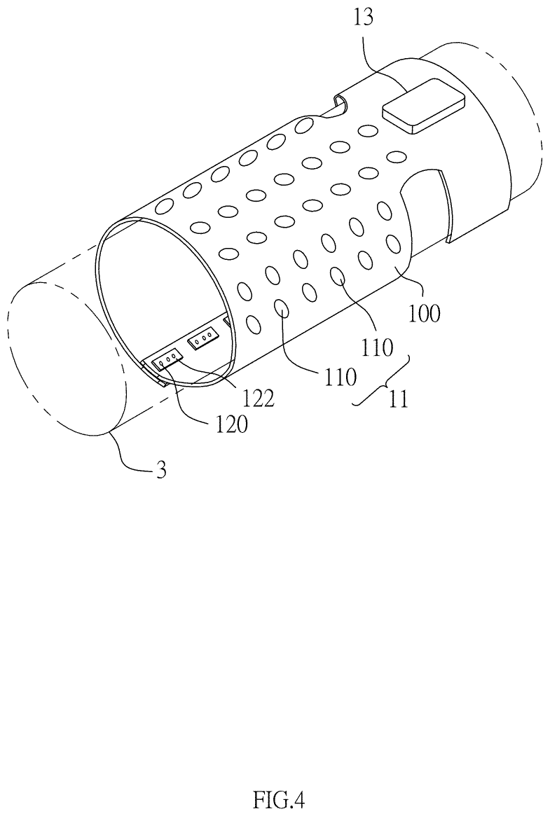

FIG. 3 and FIG. 4 show embodiments of the invention in use. FIG. 3 shows an embodiment connected or attached to a human with a belt, and FIG. 4 shows an embodiment that includes the device conformed to a body part 3, such as an arm, leg, neck, or penis. As shown in FIG. 3, belt 2 is received in holes 1020 and wrapped around a waist to be fixed, allowing the device 1 to be tied on the waist.

Embodiments of the present invention include methods of using a device as described above. Embodiments include providing a device as described above, including a device as depicted in FIG. 1 and FIG. 2 and FIG. 3, which includes an enclosing portion and a light emitting element that can include an LED array. Exemplary methods can also include attaching or fixing or connecting the device to a body with a belt. Exemplary methods can also include using means for attaching or fixing the device, such as magnets and metallic pieces, to attach or fix the enclosing portion 100 to substantially conform to the shape of a body part, such as an arm, a leg, an ankle, a neck, or a penis. Exemplary methods can also include using a controller to activate the light emitting element. Exemplary methods can also include delivering various wavelengths of light to at least a portion of a body part, such as an arm, a leg, an ankle, a neck, or a penis, the wavelengths of light including any wavelength between about 600 nm and 910 nm, RED LED (wavelength 625 nm-680 nm) and INFRA RED (wavelength 810 nm-910 nm). Exemplary methods can also include delivering light in a predetermined sequence or pattern, as well as for a pre-determined length of time.

In preferred embodiments, the base material encloses a penis and wavelengths of light are delivered to the penis to improve blood flow in and around the penis, and to improve a man's sexual ability, health, as well as to reduce symptoms of erectile dysfunction.

Certain features that are described in this disclosure in the context of separate implementations can also be implemented in combination in a single implementation. Conversely, various features that are described in the context of a single implementation also can be implemented in multiple implementations separately or in any suitable subcombination. Moreover, although features may be described above as acting in certain combinations, one or more features from a claimed combination can in some cases be excised from the combination, and the combination may be claimed as a subcombination or variation of a subcombination.

Any portion of any of the steps, processes, structures, and/or devices disclosed or illustrated in one example in this disclosure can be combined or used with (or instead of) any other portion of any of the steps, processes, structures, and/or devices disclosed or illustrated in a different example or flowchart. The examples described herein are not intended to be discrete and separate from each other. Combinations, variations, and some implementations of the disclosed features are within the scope of this disclosure.

While operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Additionally, the operations may be rearranged or reordered in some implementations. Also, the separation of various components in the implementations described above should not be understood as requiring such separation in all implementations, and it should be understood that the described components and systems can generally be integrated together in a single product or packaged into multiple products. Additionally, some implementations are within the scope of this disclosure.

Further, while illustrative examples have been described, any examples having equivalent elements, modifications, omissions, and/or combinations are also within the scope of this disclosure. Moreover, although certain aspects, advantages, and novel features are described herein, not necessarily all such advantages may be achieved in accordance with any particular example. For example, some examples within the scope of this disclosure achieve one advantage, or a group of advantages, as taught herein without necessarily achieving other advantages taught or suggested herein. Further, some examples may achieve different advantages than those taught or suggested herein.

Some examples have been described in connection with the accompanying drawings. The figures are not all drawn and/or shown to scale, but such scale should nevertheless not be limiting, since dimensions and proportions other than what are shown are contemplated and are within the scope of the disclosed inventions. Distances, angles, etc. are merely illustrative and do not limit the relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, any methods described herein may be practiced using any device suitable for performing the recited steps.

It is of course to be understood that the embodiments described herein is merely illustrative of the principles of the invention and that a wide variety of modifications thereto may be effected by persons skilled in the art without departing from the spirit and scope of the invention as set forth in the following claims.

1. A phototherapeutic device for a penis, comprising:
a flexible base material that includes a rectangular shaped enclosing portion and an elongate fixing portion, wherein the enclosing portion is configured to substantially conform to the shape of a body part;
a light emitting element disposed at least partially on the enclosing portion, wherein the light emitting element comprises a plurality of light emitting diode (LED) light sources;
means for fixing or attaching the enclosing portion to substantially conform to the shape of a body part, wherein the means for fixing or attaching the enclosing portion to conform to the shape of the body part is disposed on the enclosing portion; and
a controller, wherein the controller is disposed on the fixing portion and is operably connected to the light emitting element.

2. The phototherapeutic device for a penis according to claim 1, wherein the first fixing or attaching part is magnets disposed on one side of the enclosing portion, and the second fixing or attaching part is metallic pieces disposed on another side of the enclosing portion.

3. The phototherapeutic device for a penis according to claim 1, wherein the fixing portion includes a hole or holes configured to receive a belt.

4. The phototherapeutic device for a penis according to claim 1, further comprising a belt.

5. The phototherapeutic device for a penis according to claim 1, further comprising a translucent element disposed at least partially over the enclosing portion and at least partially covering the light emitting element.

6. The phototherapeutic device for a penis according to claim 1, wherein the LED light sources deliver wavelengths of light in the range from about 600 nm to about 910 nm.

7. The phototherapeutic device for a penis according to claim 1, wherein the flexible base material is silica gel.

8. A method of using a phototherapeutic device for a penis, comprising steps of:
a) providing a device that includes a flexible base material that includes:
a rectangular shaped enclosing portion and an elongate fixing portion,
wherein the enclosing portion is configured to substantially conform to the shape of a body part;
a light emitting element disposed at least partially over the enclosing portion, wherein the light emitting element comprises a plurality of light emitting diode light sources;
means for fixing or attaching the enclosing portion to substantially conform to the shape of the body part, wherein the means for fixing or attaching the enclosing portion to conform to the shape of a body part is disposed on the enclosing portion;
a belt; and
a controller, wherein the controller is disposed on the fixing portion and is operably connected to the light emitting element;
b) fixing or attaching the device to a human using the belt;
c) fixing or attaching the enclosing portion to substantially conform to the shape of a body part;
d) activating the light emitting element using the controller to deliver light having wavelengths between about 600 nm and about 910 nm.

9. The phototherapeutic device for a penis according to claim 1, wherein the means for fixing or attaching the enclosing portion to substantially conform to the shape of the body part includes a first fixing or attaching part and a second fixing or attaching part that is fixed or attached to the first fixing or attaching part, and
when the second fixing or attaching part is fixed or attached to the first fixing or attaching part, the enclosing portion forms a cylindrical shape.

10. The method according to claim 1, wherein the means for fixing or attaching the enclosing portion to substantially conform to the shape of the body part includes a first fixing or attaching part and a second fixing or attaching part that is fixed or attached to the first fixing or attaching part, and
fixing or attaching the enclosing portion includes fixing or attaching the second fixing or attaching part to the first fixing or attaching part to form the enclosing portion in a cylindrical shape.

11. The phototherapeutic device for a penis according to claim 1, wherein the light delivered by the LED light sources include at least one of red light having a wavelength in a range of 625 nm-680 nm and infrared light having a wavelength in a ranging of 810 nm-910 nm.

12. The method according to claim 8, wherein the light delivered by the light emitting element include at least one of red light having a wavelength in a range of 625 nm-680 nm and infrared light having a wavelength in a ranging of 810 nm-910 nm.
